(19) United States
(12) Patent Application Publication
(10) Pub. No.: US 2021/0402021 A1
(43) Pub. Date: Dec. 30, 2021

(54) Ultraviolet light equipment rack

(71) Applicant: Nemesis UVC LLC, Garland, TX (US)

(72) Inventors: Michael Nevitt, Garland, TX (US); Eric L. Wade, Garland, TX (US); John Early, Garland, TX (US)

(21) Appl. No.: 17/320,609

(22) Filed: May 14, 2021

Related U.S. Application Data
(60) Provisional application No. 63/024,878, filed on May 14, 2020.

(51) Int. Cl. A61L 2/10

(52) U.S. Cl. CPC A61L 2/10; A61L 2202/11; A61L 2202/16; A61L 2202/122; A61L 2202/14

(57) ABSTRACT

A portable ultraviolet (UV) light rack for use in preventing microbial or pathogen growth within or on various types of equipment, such as sports equipment. The UV light equipment rack is configured to disinfect multiple pieces of equipment, such as basketballs, football helmets, or non-sport equipment, such as masks. The UV light equipment rack may include an upper frame, a lower frame, a first side frame, and a second side frame. The interior portion of the UV light equipment rack is configured to receive and hold or secure the equipment to be disinfected via UV light, and to provide one or more disinfecting systems, such as UV light systems. The UV light equipment rack may include one or more equipment holding assemblies and one or more UV light panels.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/024,878, entitled “ULTRAVIOLET LIGHT EQUIPMENT RACK” filed May 14, 2020. The contents of the above referenced application are incorporated herein by reference in its entirety.

### FIELD OF THE INVENTION

The present invention relates to devices, systems, and methods for disinfecting equipment; more particularly, to portable ultraviolet (UV) racks for use in preventing microbial or pathogen growth on or within one or more types of equipment.

### BACKGROUND OF THE INVENTION

The sports industry is a multibillion dollar industry deeply rooted in most societies. Like most businesses, most, if not all, sports related businesses have been profoundly affected by the 2019-2020 COVID 19 outbreak. During the pandemic, many societies ordered shelter in place strategies to prevent rapid spread of the disease. With no cure available and long term vaccine effectiveness currently unavailable, non-pharmacological means must be employed to prevent further spread. As most industries begin to open up and head towards normal business practices, the new normal includes close attention to sanitization efforts to provide clean and safe environments.

The sports industry faces many of the same problems as other industries when seeking to provide safe and clean environments for their customers. Unlike other industries, however, many sport businesses have unique issues related to those participating in the sports, particularly those sports that require use of equipment or are team based. Sports such as basketball or football that require special equipment, such as football helmets or basketballs, must ensure such equipment is ready for use. As these team sports require participants to engage in close contact, social distancing is not an option and the risk of transmitting the disease from player to player is great. Various sports equipment, such as football helmets, footballs and basketballs are not very easy to sanitize, and washing them with soap and water or other liquid sanitation compositions may cause damage. Devices for sanitizing such equipment, particularly devices that utilize Ultraviolet (UV) light systems that disinfect quickly and safely, are needed.

### SUMMARY OF THE INVENTION

Embodied are devices, systems, and methods for disinfecting equipment. Particularly, the invention is directed towards portable ultraviolet (UV) racks for use in preventing microbial or pathogen growth within one or more type of equipment, such as sports equipment. An illustrative embodiment of a UV light based disinfection device, preferably a UV light equipment rack, is configured to disinfect multiple pieces of equipment, such as basketballs, football helmets, or non-sport equipment such as masks. The UV light equipment rack may include an upper frame, a lower frame, a first side frame, and a second side frame. The interior portion of the UV light equipment rack is configured to receive and hold or secure the equipment to be disinfected via UV light, and to provide one or more disinfecting systems, such as UV light systems. The UV light equipment rack may include one or more equipment holding assemblies and one or more UV light panels.

Accordingly, it is an objective of the invention to provide a unique disinfection device.

It is a further objective of the invention to provide a disinfection device using UV light.

It is yet another objective of the invention to provide a disinfection device configured to sanitize equipment.

It is a still further objective of the invention to provide a UV light based disinfection device configured to sanitize sports equipment.

It is a further objective of the invention to provide a UV light based disinfection device configured to sanitize sports equipment, such as football helmets.

It is yet another objective of the invention to provide a UV light based disinfection device configured to sanitize sports equipment, such as basketballs.

It is a still further objective of the invention to provide a UV light based disinfection device configured to sanitize equipment, such as masks.

It is a further objective of the invention to provide a UV light based disinfection device configured to sanitize sports equipment which uses a rail system.

It is yet another objective of the invention to provide a UV light based disinfection device configured to sanitize sports equipment which uses an adjustable rail system.

It is yet another objective of the invention to provide a UV light based disinfection device configured to sanitize sports equipment which uses a removable rail system.

It is a further objective of the invention to provide a UV light based disinfection device configured to sanitize sports equipment which uses spring clips.

It is yet another objective of the invention to provide a UV light based disinfection device configured to sanitize sports equipment which is portable.

It is a still further objective of the invention to provide a UV light based disinfection device configured to sanitize sports equipment which uses an in-use warning system to prevent unwanted UV light exposure to the user.

It is a further objective of the invention to provide a UV light based disinfection device configured to sanitize sports equipment which uses an LED beacon in-use warning light.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1A is a front side perspective view of an illustrative embodiment of a UV light based disinfecting equipment rack, shown with basketballs inserted thereon;

FIG. 1B is a front side perspective view of the UV light based disinfecting equipment rack, shown with enclosed side walls;

FIG. 1C is a front view of the UV light based disinfecting equipment rack with the enclosed side walls;

FIG. 2 is a back side perspective view of the UV-light based disinfecting equipment rack;

FIG. 3 is a front view of the UV light based disinfecting equipment rack;

FIG. 4A is a front view of the UV light based disinfecting equipment rack, with the basketballs removed;

FIG. 4B is a back view of the UV light based disinfecting equipment rack, with the basketballs removed;

FIG. 5A is a first side view of the UV light based disinfecting equipment rack, with the basketballs removed;

FIG. 5B is a second side view of the UV light based disinfecting equipment rack, with the basketballs removed;

FIG. 6 is an illustrative embodiment of a UV light panel;

FIG. 7A illustrates the UV light based disinfecting equipment rack with an in-use cover;

FIG. 7B illustrates the UV light based disinfecting equipment rack with closed ends, shown with the in-use cover;

FIG. 8 is a front side perspective view of the UV light based disinfecting equipment rack shown with football helmets placed thereon;

FIG. 9 is a back side perspective view of the UV light based disinfecting equipment rack shown in FIG. 8;

FIG. 10 is a front view of the UV light based disinfecting equipment rack shown in FIG. 8;

FIG. 11 illustrates the UV light based disinfecting equipment rack shown in FIG. 8 placed within a locker room;

FIG. 12 illustrates the UV light based disinfecting equipment rack shown in FIG. 11, in use with a cover;

FIG. 13 is a front side perspective view of the UV light based disinfecting equipment rack shown with masks attached thereto;

FIG. 14 is a back side perspective view of the UV light based disinfecting equipment rack shown in FIG. 13 with the masks removed;

FIG. 15 is a front view of the UV light based disinfecting equipment rack shown in FIG. 13 with the masks removed;

FIG. 16 illustrates the UV light based disinfecting equipment rack with a side to side roller shade enclosure;

FIG. 17 illustrates the UV light based disinfecting equipment rack shown in FIG. 16 with the shade partially extended;

FIG. 18 illustrates the UV light based disinfecting equipment rack with a top to bottom roller shade enclosure;

FIG. 19 illustrates the UV light based disinfecting equipment rack shown in FIG. 18 with the shade partially extended;

FIG. 20 illustrates the UV light based disinfecting equipment rack with baseball helmets inserted therein;

FIG. 21 is a perspective view of a smaller version of the UV light based disinfecting equipment rack;

FIG. 22 illustrates the UV light based disinfecting equipment rack shown in FIG. 21 in an open position;

FIG. 23 illustrates the UV light based disinfecting equipment rack shown in FIG. 21 with baseballs and baseball bats inserted therein; and

FIG. 24 illustrates a tabletop version of the UV light based disinfecting equipment rack.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

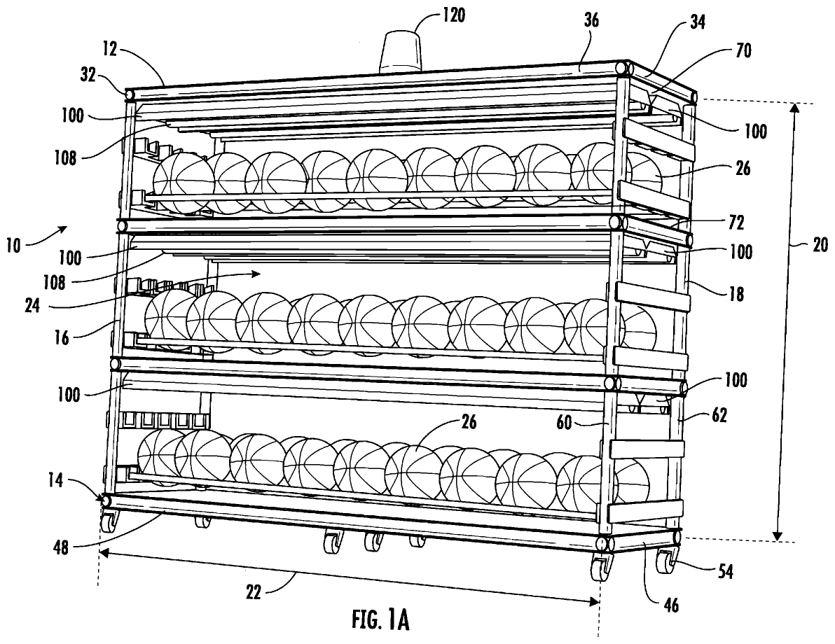

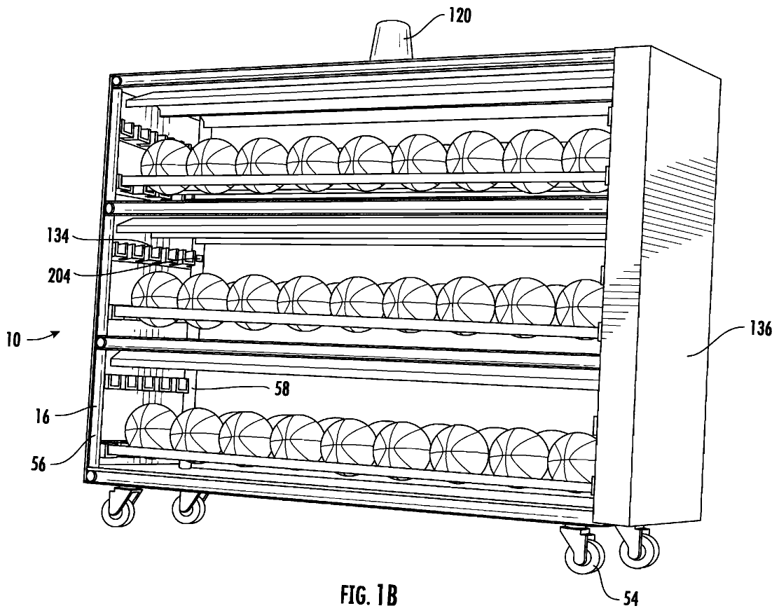

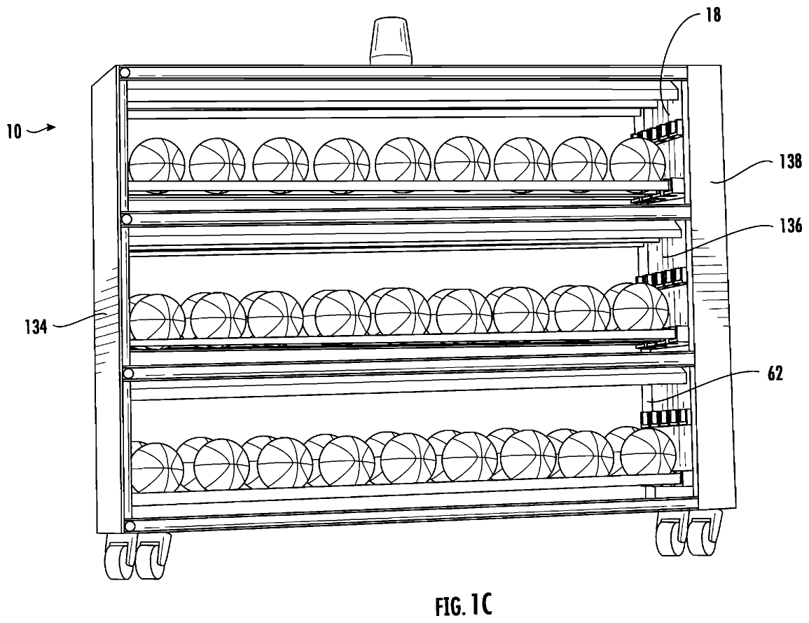

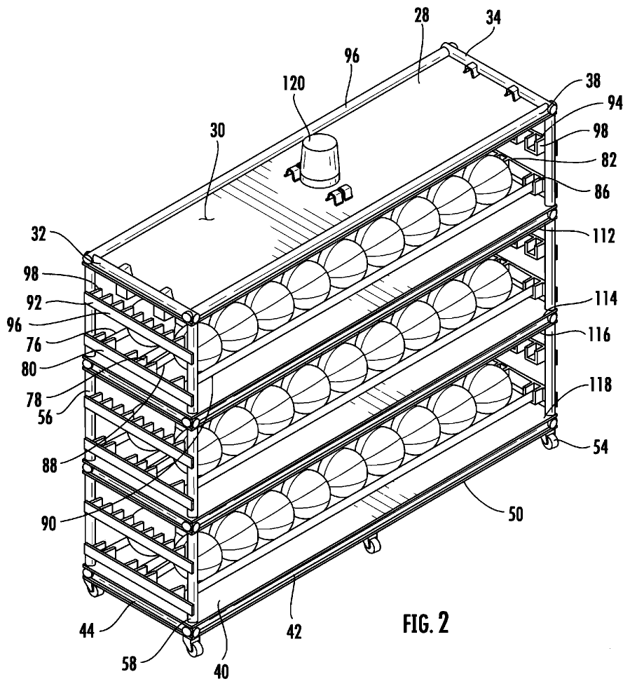

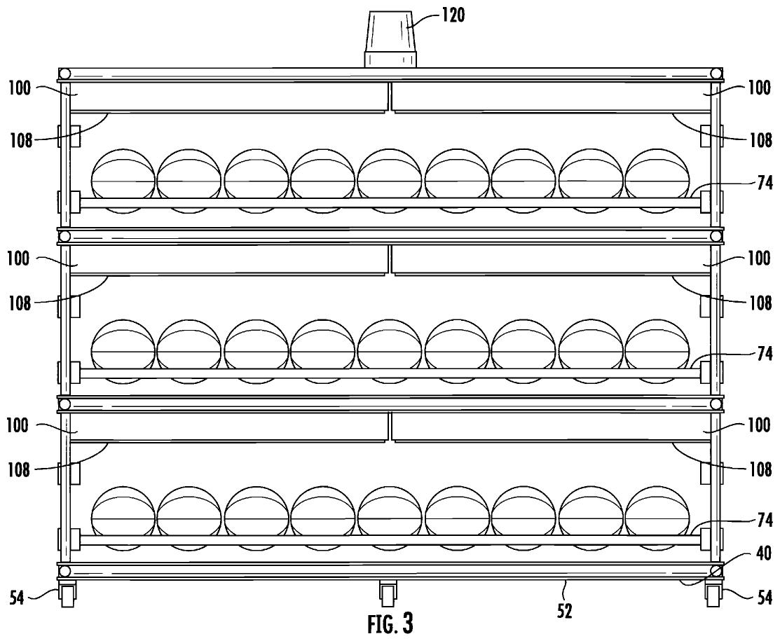

Referring to FIGS. 1A-3, an illustrative embodiment of a UV light based disinfection device, referred to generally as a UV light equipment rack 10, is shown. The UV light equipment rack 10 may be powered using an electrical power cord for connection to an electrical outlet, or may be powered by a battery. The UV light equipment rack 10 is configured to disinfect multiple pieces of equipment, such as sports equipment, simultaneously during a single run or time period. The UV light equipment rack 10 is defined by an upper frame 12, a lower frame 14, a first side frame 16, and a second side frame 18. The UV light equipment rack 10 may be made of a metal material with a highly reflective white powder, preferably electrostatically applied, coating. Alternatively, the UV light equipment rack 10 may be coated with a highly reflective paint. The upper frame 12 and the lower frame 14 are separated by a space defining a vertical distance 20, and are arranged in a generally parallel manner relative to each other. The first side frame 16 and the second side frame 18 are separated by a space defining a horizontal distance 22, and are arranged in a generally parallel manner relative to each other. The upper frame 12, the lower frame 14, the first side frame 16, and the second side frame 18 are secured together to define an interior 24. The interior 24 is designed to receive and store therein one or more pieces of equipment, such as sports equipment, illustrated herein as a plurality of basketballs 26. While the embodiment shown in FIGS. 1A-3 illustrates sports equipment as a plurality of basketballs 26, the UV light equipment rack 10 can hold other balls, such as football and soccer balls, other sports equipment, such as football or baseball helmets, and other types of equipment other than sports equipment, such as masks.

The upper frame 12 may be defined by a top plate 28 defining the UV light equipment rack 10 upper surface 30 and surrounded along its perimeter by posts or two short open side walls 32 and 34, and two long posts or open side walls 36 and 38. The lower frame 14 may be defined by a bottom plate 40 defining the UV light equipment rack 10 lower surface 42 and surrounded along its perimeter by posts or two short open side walls 44 and 46, and two long posts or open side walls 48 and 50. Secured to the bottom surface 52 of the bottom plate 40 are one or more wheel assemblies, illustrated herein as castor wheels 54. The castor wheels 54 are spaced sufficiently apart to provide balance and allow the UV light equipment rack 10 to be quickly and easily moved from one place to another.

The first side frame 16 comprises two elongated vertical bars or bodies 56 and 58 extending from and secured to (via welding) or integrally formed from the upper frame 12 and secured to or integrally formed from the lower frame 14. The first side frame elongated vertical bars or bodies 56 and 58 are spaced apart and arranged in a generally parallel manner relative to each other. The second side frame 18 comprises two elongated vertical bars or bodies 60 and 62 extending from and secured to (via welding) or integrally formed from the upper frame 12 and secured to or integrally formed from the lower frame 14. The second side frame elongated vertical bars or bodies 60 and 62 are spaced apart and arranged in a generally parallel manner relative to each other.

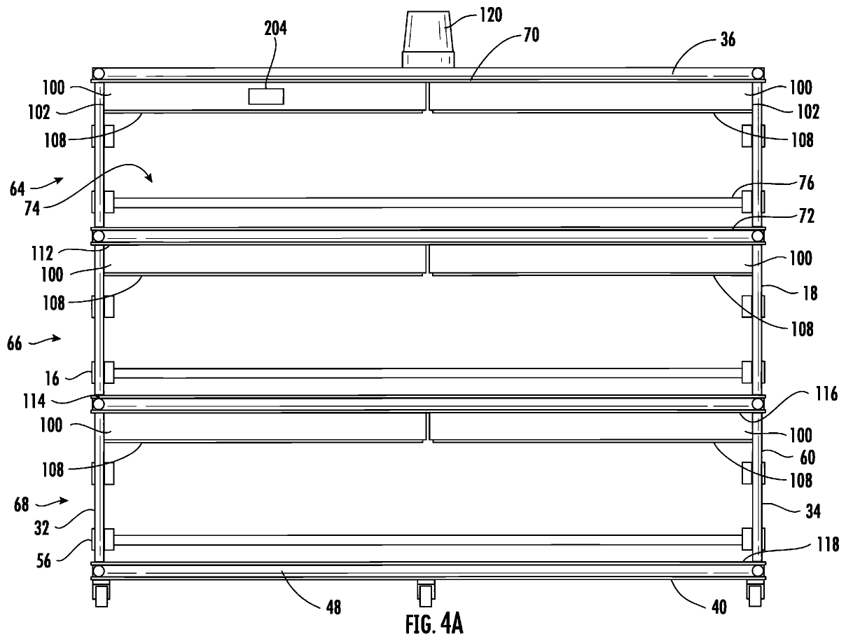

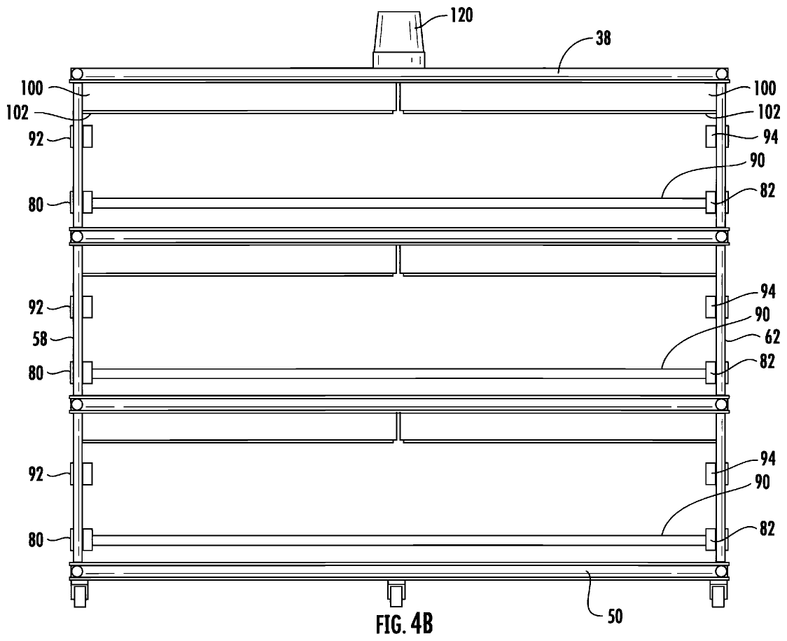

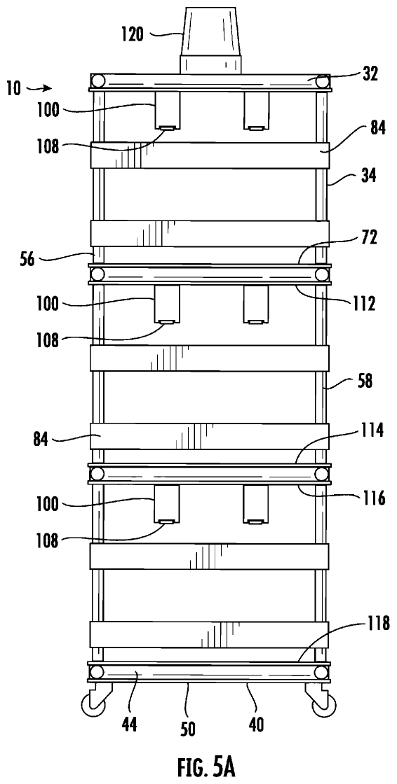

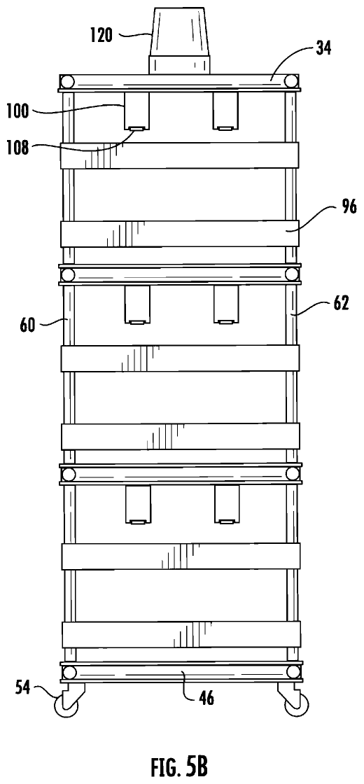

The interior portion 24 is configured to 1) receive and hold or secure one or more pieces of equipment to be disinfected via UV light, and 2) contain one or more disinfecting systems, such as UV light systems. Accordingly, the interior portion 24 can be sub-divided into individual equipment holding/disinfecting areas or zones. As shown in FIGS. 1A-5B, the interior portion 24 is divided into three equipment holding/disinfecting areas or zones 64, 66, and 68, see FIG. 4A with basketballs removed. The equipment holding/disinfecting zone 64 may comprise a top plate 70 and a bottom plate 72. The top plate 70 spans the distance from the first side frame 16 to the second side frame 18, and is secured (welded) to or integrally formed as at least a portion of the first side frame 16 and the second side frame 18. The bottom plate 72 spans the distance from the first side frame 16 to the second side frame 18, and is secured (welded) to or integrally formed as at least a portion of the first side frame 16 and the second side frame 18.

The equipment holding/disinfecting zone 64 may further include one or more equipment holding assemblies 74 designed to hold one or more items, such as sports equipment. The equipment holding assemblies 74 may include one or more equipment holding members configured to hold a piece of equipment, i.e. the basketball, illustrated herein as a first pair of elongated bars 76 and 78, each secured to a first lower bracket 80 at one end and to a second lower bracket 82 at a second end. The first lower bracket 80 is secured to at least a portion of the first side frame 16. The second lower bracket 82 is secured to at least a portion of the second side frame 18. The first lower bracket 80 and the second lower bracket 82 each comprise a cross bar body 84 with a plurality of elongated bar receiving members, illustrated herein as U-shaped brackets 86, each sized and shaped to receive and hold the first pair of elongated bars 76 and 78 when placed therein. Each of the elongated bars 76 and 78 are arranged in a generally parallel orientation relative to each other, and spaced apart by a sufficient distance or space to accommodate and hold the specific equipment being applied thereto.

The equipment holding assemblies 74 may further include a second pair of elongated bars 88 and 90, each secured to the first lower bracket 80 at one end and to the second lower bracket 82 at a second end as described above, and having the same features as described above for the first pair of elongated bars 76 and 78.

In order to add additional capacity and to adjust for differently sized pieces of equipment, the equipment holding assemblies 74 may include a first upper bracket 92 and a second upper bracket 94. The first upper bracket 92 is secured to at least a portion of the first side frame 16. The second upper bracket 94 is secured to at least a portion of the second side frame 18. The first upper bracket 92 and the second upper bracket 94 each comprise a cross bar body 96 with a plurality of elongated bar receiving members, illustrated herein as U-shaped brackets 98, each sized and shaped to receive and hold the second pair of elongated bars 88 and 90 when placed therein. In some embodiments, the elongated bar receiving members illustrated as a row(s) of U-shaped brackets 98 may be secured to or integrally formed within the first enclosed side wall or surface 134 or the second enclosed side wall or surface 136.

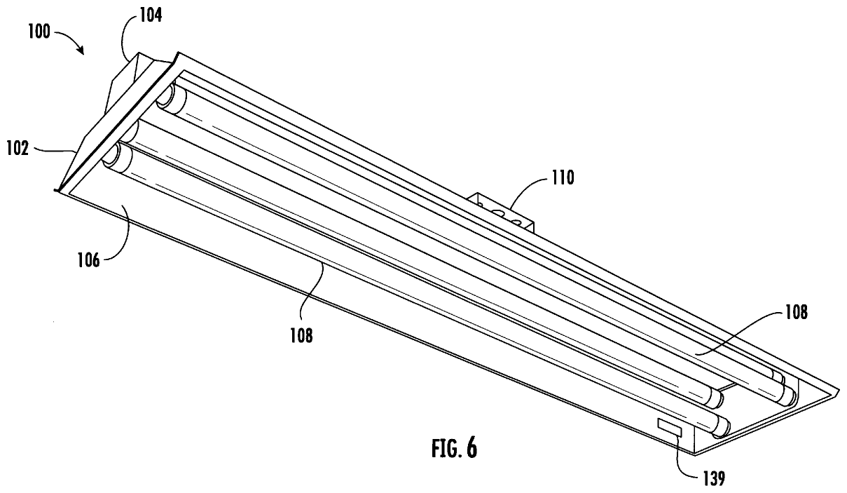

Attached or secured to the equipment holding/disinfecting zone top plate 70 is one or more disinfecting units, illustrated herein as UV light panels 100. FIG. 6 provides an illustrative example of the UV light panel 100. The UV light panel 100 may include a source housing 102, an electrical housing 104, which may include the electrical hardware and ballast (not shown), a reflector 106, an LED UV radiator or lamp 108, and a bracket 110 for securing to the equipment holding/disinfecting zone top plate 70. The UV light panel 100 is preferably secured to the equipment holding/disinfecting zone top plate 70 via mechanical fastening members such as screws and bolts. The UV light panel 100 may include a power cord (not shown) for plugging into an electrical outlet or an electrical outlet bank. Preferably, the UV light panel 100 utilizes one or more LED UV-C bulbs, (emitting in the range of 200-400 nm, preferably in the range of approximately 254 nm to about 280 nm), preferably UV-C LEDs. While described as using UV-C light, other UV lights that have the ability to inactivate pathogens (both viruses and bacteria) by impacting the cellular RNA and DNA, damaging nucleic acids, and preventing microorganisms from infecting and reproducing, or acting as a disinfectant may be used as well.

Although not illustrated, additional UV light panels 100 may be secured to the equipment holding/disinfecting zone 64 bottom plate 70. If multiple UV light panels 100 are used, the UV light equipment rack 10 may be configured so that when powered ON (OFF), all UV light panels 100 turn ON (OFF) and emit light at the same time, or each individual UV light panel 100 may be turned ON (OFF) and emit light separately and independently. Each individual UV light panel 100 may be configured to be separately removably mountable, such as via an adjustable bracket, to the UV light equipment rack 10. Such functionality allows a user to add or remove the UV light panels 100 as needed.

The equipment holding/disinfecting zone 66 may comprise a top plate 112 and a bottom plate 114. The top plate 112 spans the distance from the first side frame 16 to the second side frame 18, and is secured (welded) to or integrally formed as at least a portion of the first side frame 16 and the second side frame 18. The bottom plate 114 spans the distance from the first side frame 16 to the second side frame 18, and is secured (welded) to or integrally formed as at least a portion of the first side frame 16 and the second side frame 18. The equipment holding/disinfecting zone 66 may further include one or more equipment holding assemblies 74 having all the same features as described above. The equipment holding/disinfecting zone 66 may include one or more disinfecting units, illustrated herein as UV light panels 100, as described above.

The equipment holding/disinfecting zone 68 may comprise a top plate 116 and a bottom plate 118. The top plate 116 spans the distance from the first side frame 16 to the second side frame 18, and is secured (welded) to or integrally formed as at least a portion of the first side frame 16 and the second side frame 18. The bottom plate 118 spans the distance from the first side frame 16 to the second side frame 18, and is secured (welded) to or integrally formed as at least a portion of the first side frame 16 and the second side frame 18. The equipment holding/disinfecting zone 68 may further include one or more holding assemblies 74 having all the same features as described above. The equipment holding/disinfecting zone 68 may include one or more disinfecting units, illustrated herein as UV light panels 100, as described above.

While the UV light equipment rack 10 is shown with three equipment holding/disinfecting zones, 64, 66, and 68, the UV light equipment rack 10 may include one, two, or more than three. Additionally, UV light panels 100 may include a source housing 102 having slots or openings in the top of sufficient size to allow passage of emitted light. In this embodiment, the UV light panels 100 would be configured to multi-directional light passage, i.e. directed in a downward direction (from the top, towards the basketballs secured in the current equipment holding/disinfecting zone) and in an upward direction (towards the bottom of the basketballs secured in an equipment holding/disinfecting zone above). To allow UV light to be emitted or directed to a zone above, the adjacent equipment holding/disinfecting zone bottom plate 72 may also be configured with slots or openings to allow passage of the UV light.

To prevent accidental UV light exposure, the UV light equipment rack 10 may include an in-use warning system. The in-use warning system may include an LED beacon in-use warning light 120, see FIG. 1. The LED beacon in-use warning light 120 is operatively connected to the UV light panels 100. Accordingly, when the UV light equipment rack 10 is exposing the air, surfaces, or the equipment stored therein to UV-C light, the LED beacon in-use warning light 120 is turned on automatically. As soon as the UV light is turned off, the LED beacon in-use warning light 120 automatically turns off. The LED beacon in-use warning light 120 may be configured to flash and have an audio alarm. The beacon in-use warning light 120 preferably has a separate power cord (not shown) to be plugged into a separate electrical outlet (not shown).

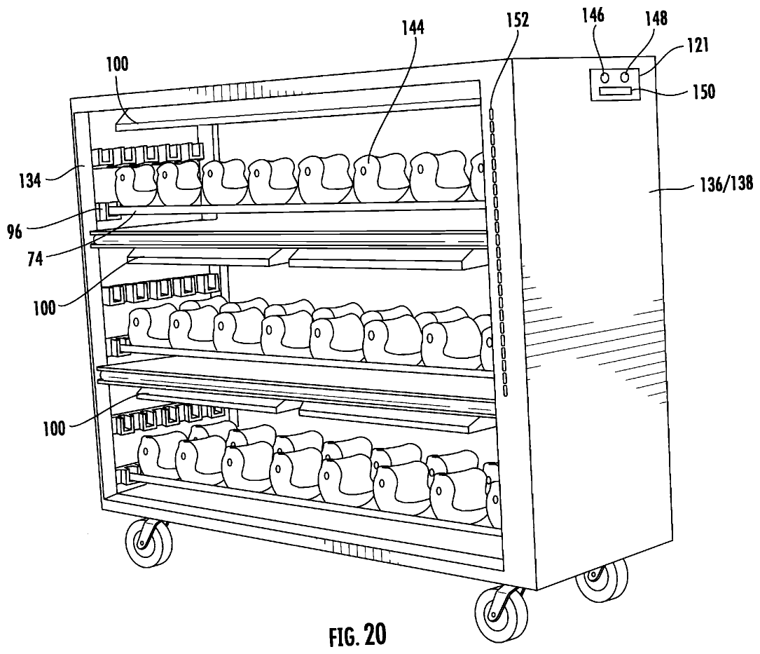

The UV light equipment rack 10 may include a WIFI switch with power cord (not shown), such as a SONOFF WIFI connector, to be used to turn all UV light panels 100 “ON/OFF” via an app. Alternatively, the UV light equipment rack 10 may be RF (radio frequency) compatible and include an RF key fob for quick on/off functioning. The UV light equipment rack 10 may be configured to operate using other wireless technologies, such as BLUETOOTH. The UV light equipment rack 10 may be configured to be a smart device, and include the necessary hardware and/or software. As such, any one of the features of the UV light equipment rack 10 may be controlled or operated remotely. The UV light equipment rack 10 may be configured to communicate with an electronic device, such as a smart phone or computer tablet, i.e. IPAD. Control of the UV light functioning, i.e. on/off, scheduling of on/off times, UV light run times, when each of the individual UV light panels 100 turns on/off may also be accomplished using a cell phone or other remote electronic device. If needed, the UV light equipment rack 10 may include a control panel 121, see FIG. 20 (including a processor, memory, and other hardware or software) with display, operatively connected to one or more structures to drive functionality, allowing the user to turn on/off UV lights, turn off audio or visual alarms, or program on/off times or cycles by touch screen or use of buttons.

The multiple UV light panels 100 are preferably operatively and electrically connected together so that when the UV light equipment rack 10 is powered on (either directly when UV light equipment rack 10 power cord is plugged into an electrical outlet, or when a power ON/OFF button is pressed), the individual UV light panels 100 provide UV light at the same time. In an illustrative embodiment, each of the individual UV light panel power cords may be plugged into a central power outlet bank, allowing each to turn on/off simultaneously when the UV light equipment rack 10 is turned on/off. All the electrical hardware required, including power cord(s), electrical outlet receivers, and other components for functionality, such as processors or microchips with memory, wireless technology such as a WIFI/RF/BLUETOOTH receivers and/or transmitters, may be placed within the UV light equipment rack 10 or within one of the walls or structures that make up or define the UV light equipment rack 10.

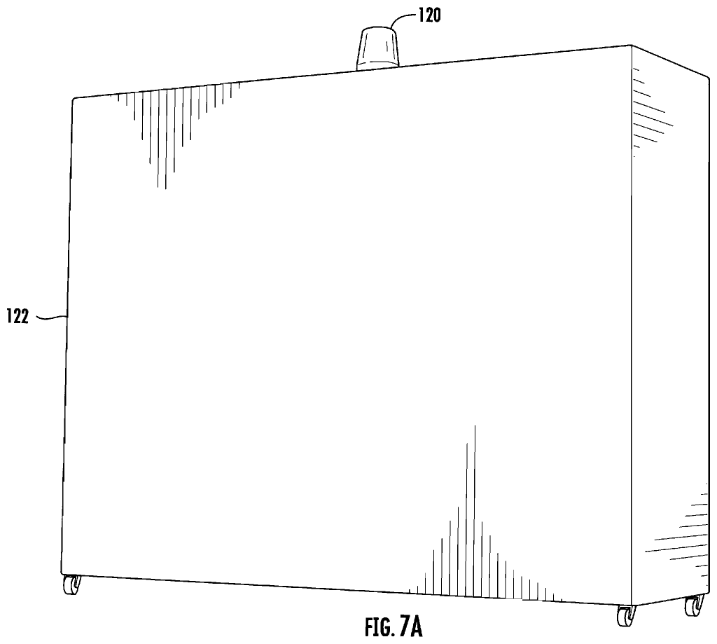

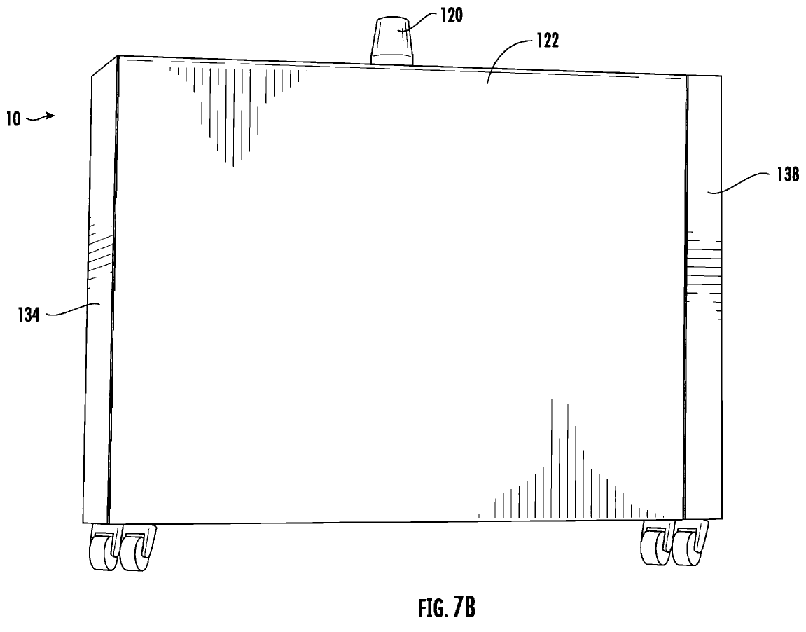

To prevent exposure of UV light to areas other than to the contents placed within or stored on the UV light equipment rack 10, a cover or drape 122, see FIGS. 7A-7B, may be placed over and secured to the UV light equipment rack 10. The drape 122 may be secured using mechanical fastening members, such as snaps, buttons, or zippers. Alternatively, the drape 122 may be secured using hook and loop fasteners, such as VELCRO.

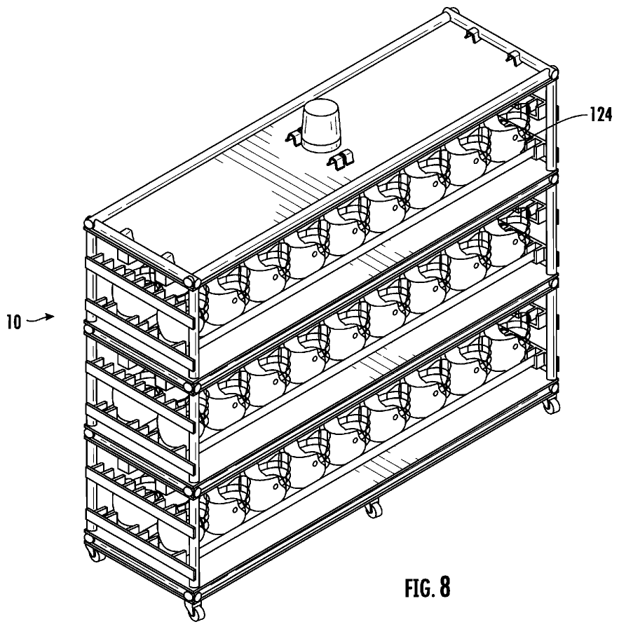

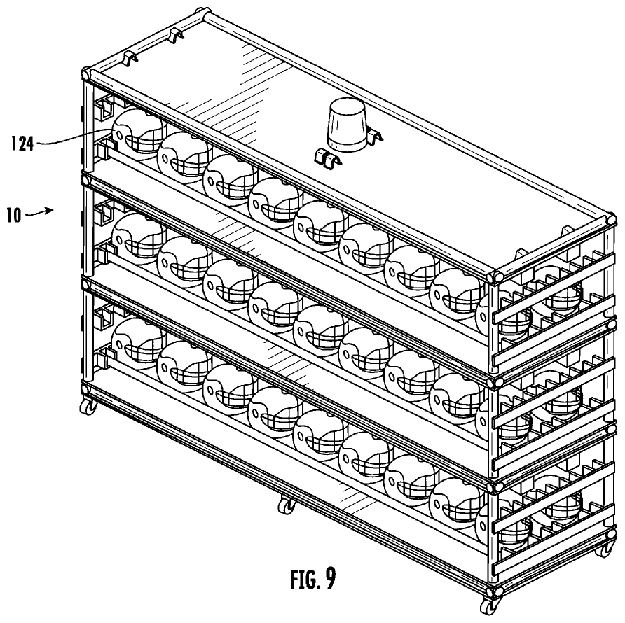

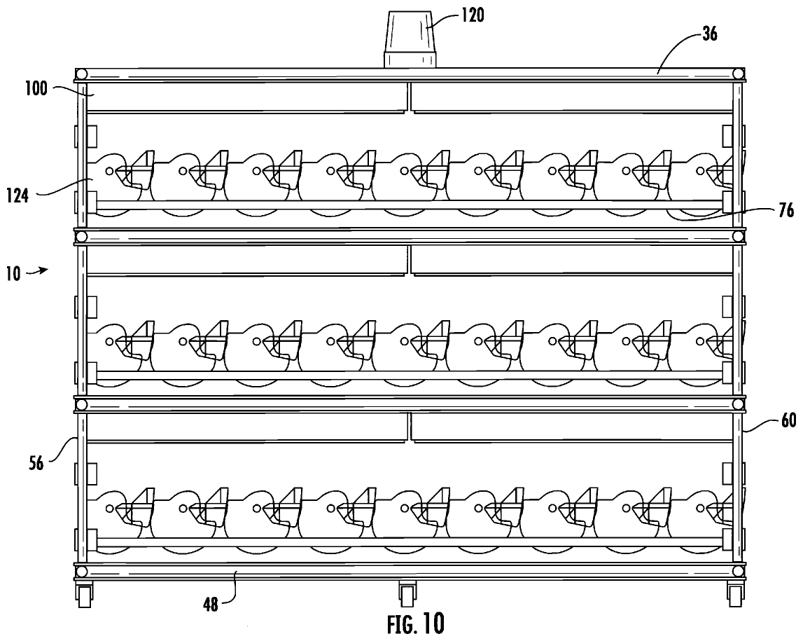

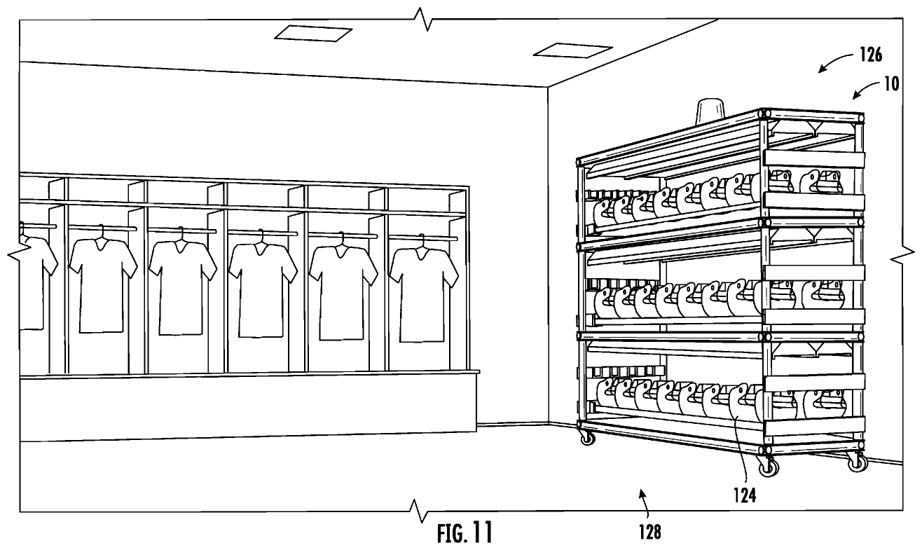

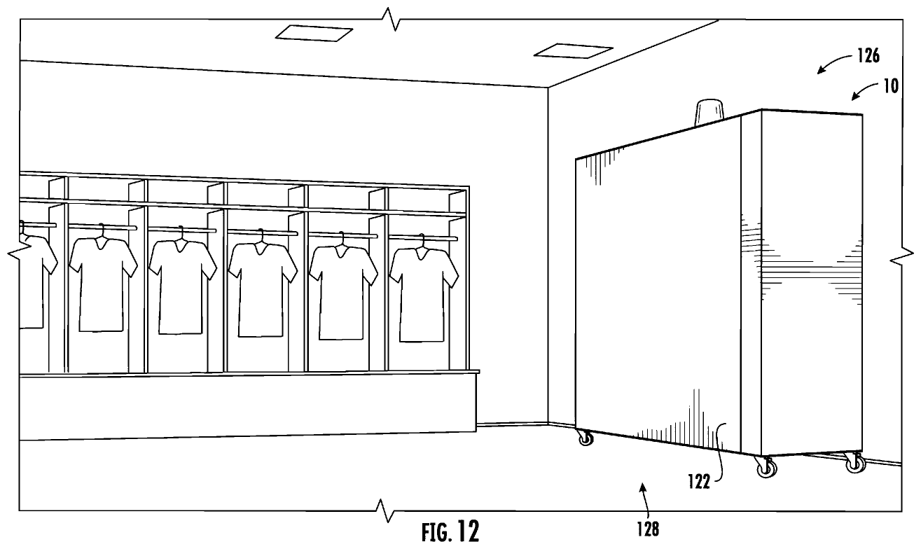

Referring to FIGS. 8-10, the UV light equipment rack 10 is shown with a plurality of football helmets 124 inserted or placed therein and resting on the equipment holding assemblies 74. FIG. 11 illustrates the UV light equipment rack 10 with football helmets 124 resting against a wall 126 of a locker room 128. FIG. 12 illustrates the UV light equipment rack 10 in operation in the locker room 128. In this operation period, the UV light equipment rack 10 with football helmets 124 is covered with a cover or drape 122 while the football helmets are being disinfected by being exposed to UV, preferably UV-C light.

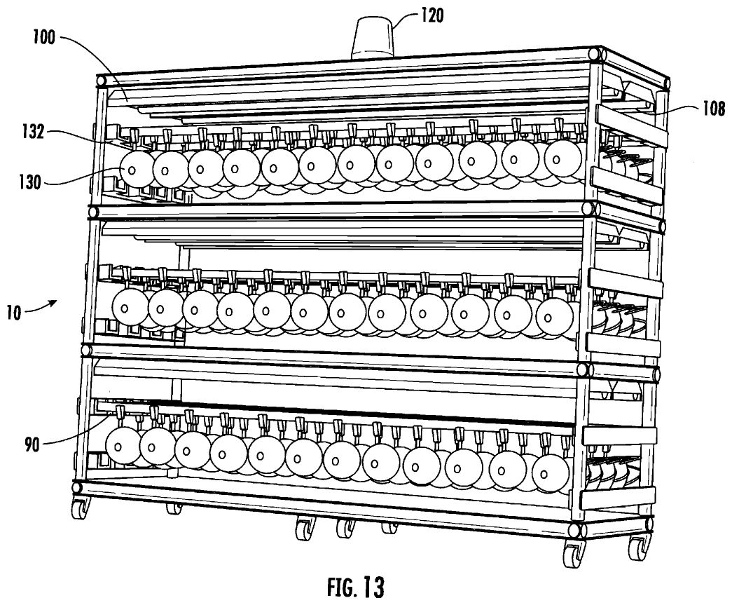

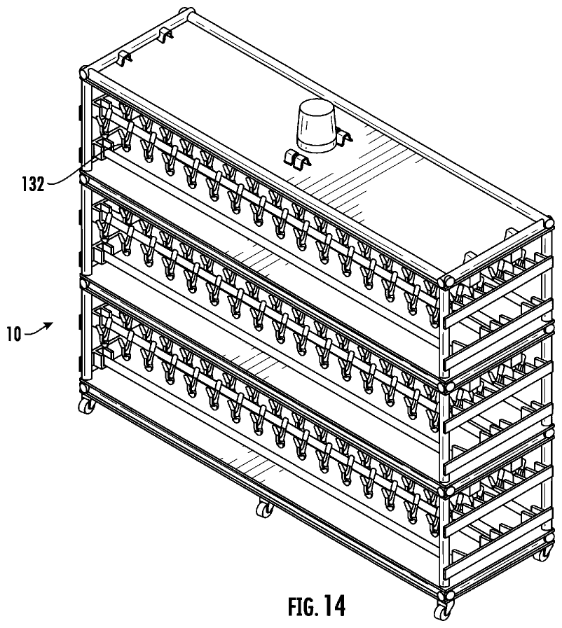

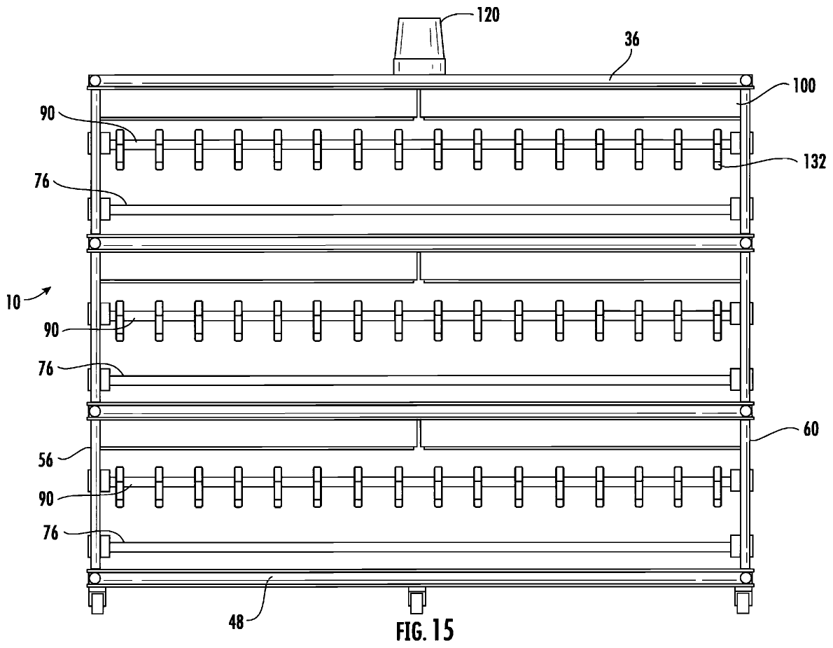

Referring to FIG. 13, the UV light equipment rack 10 is shown with a plurality of masks 130 inserted and secured to a portion of the equipment holding assembly 74. As illustrated, the masks 130 are secured to portions of the equipment holding assembly 74 via spring clips 132. FIGS. 14 and 15 illustrate the UV light equipment rack 10 without the masks 130 attached.

Referring to FIG. 1B, the UV light equipment rack 10 is shown with the first side frame 16 and the second side frame 18 enclosed. The first side frame 16 is shown with a first enclosed side wall or surface 134 and spanning the distance or space between elongated vertical bars 56 and 58. The enclosed side wall or surface 134 may be removably attached or secured to at least a portion of the upper frame 12 or lower frame 14. The enclosed side wall or surface 134 may be made as a single panel or as a plurality of individual panels so that a user may remove one of the plurality of individual panels if, for example, one of the individual equipment holding/disinfecting areas or zones (such as 64, 66, or 68), is not holding basketballs, and therefore may not need to be exposed to UV light.

Referring to FIG. 1C, the second side frame 18 is shown with a second enclosed side wall or surface 136 and spanning the distance or space between elongated vertical bars 60 (covered) and 62. The enclosed side wall or surface 136 may be removably attached or secured to at least a portion of the upper frame 12 or lower frame 14. The enclosed side wall or surface 136 may be made as a single panel or as a plurality of individual panels so that a user may remove one of the plurality of individual panels if, for example, one of the individual equipment holding/disinfecting areas or zones (such as 64, 66, or 68), is not holding basketballs, and therefore may not need to be exposed to UV light.

The UV light equipment rack 10 may include an electronic box 138 secured to the second enclosed side wall or surface 136. The electronic box 138, which may be integrated into or be part of the second enclosed side wall or surface 136, may be sized to run the entire length or distance between the upper frame 12 and lower frame 14, or any size in between. The enclosed inner region of the electronic box 138 is sized and shaped to hold one or more functional components that drive the UV light equipment rack 10, such as the electrical components, such as an LED beacon in-use warning light electrical cord, UV light panel 100 cord(s), UV light equipment rack 10 power cord, electrical connectors such as True 1 plugs (Neutrik) that allow all UV lights to power up and run simultaneously as a single unit, control panels or circuit boards, processors, memory, electrical outlet receptacles, power cord receptacles, timer units, fuses, terminal blocks, transformers, or WIFI switches (not shown), wireless technology components such WIFI/RF/BLUETOOTH transmitters and/or receivers. The UV light equipment rack 10 may also be configured so the electronic box 138 forms part of the entire second enclosed side wall or surface 136.

The UV light equipment rack 10 may include one or more occupancy sensor(s) (see 139, see FIG. 6, part of the UV light panel 100) such as motion sensors or heat/thermal sensors, within the disinfection range (air or surface, or area being exposed to the UV light) configured to immediately shut off the UV light equipment rack 10 (i.e. stop the UV lights 108 from operating) if a person or animal is within the disinfection range or opens an enclosed UV light equipment rack 10 while the UV light equipment rack 10 is in the “ON” or operating position, thus preventing accidental human/animal UV light exposure when the UV lights are operating. While the sensor 139 may be part of or integrated into the one or more UV light panels 100, the sensor 139 may additionally or alternatively be incorporated into any part of the UV light equipment rack 10. In one embodiment, each of the one or more UV light panels 100 may include its own occupancy sensors 139. All of the one or more UV light panels 100 may be turned off simultaneously, or each individual one or more UV light panels 100 may be turned off independently relative to each other.

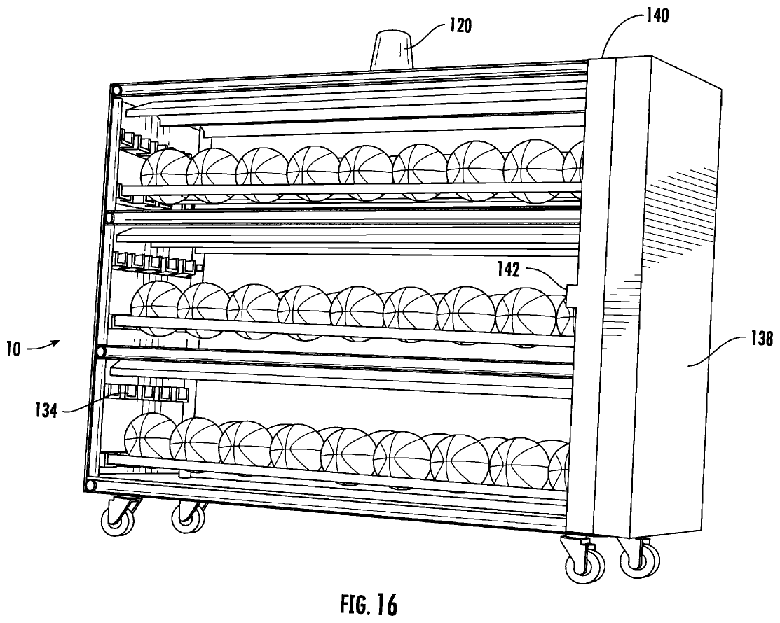

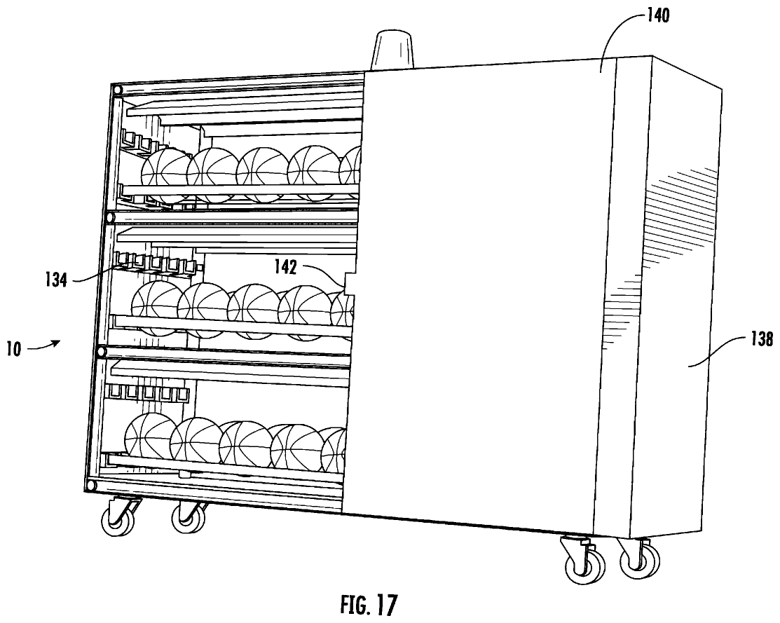

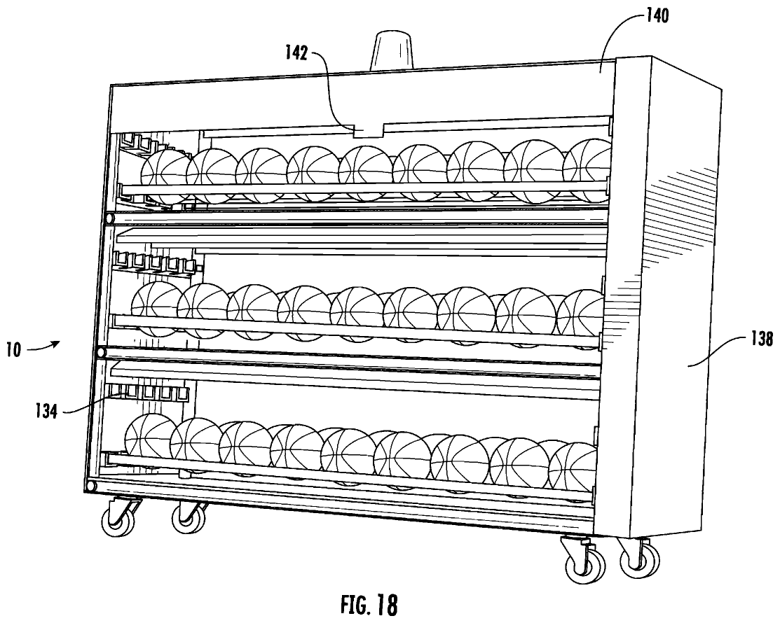

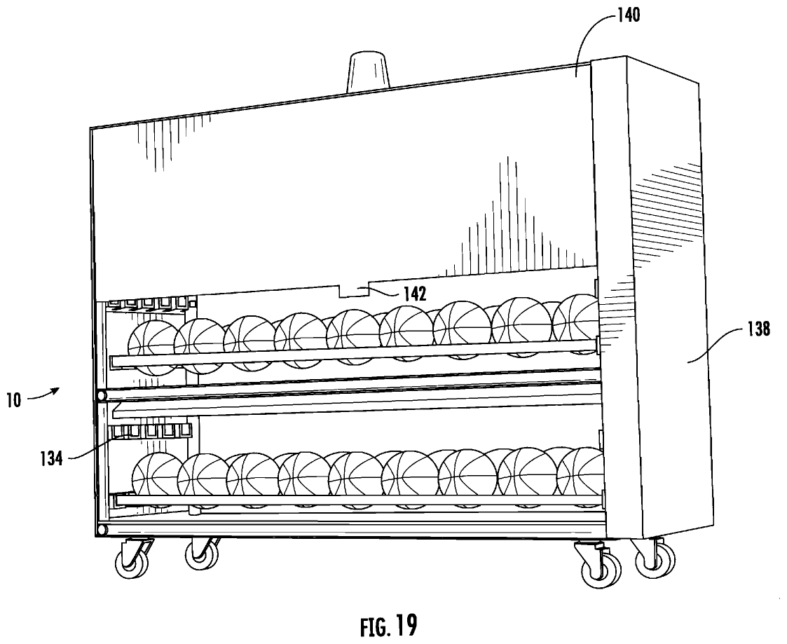

Referring to FIG. 7B, the UV light equipment rack 10 is shown with the cover or drape 122 secured (via VELCRO or other securing means as described previously) to the first enclosed side wall or surface 134 at one end and to the electronic box 138 at another end. Alternative to the cover or drape 122, the UV light equipment rack 10 may include a roller blind or shade 140 (wrapped around a reel, and may have automatic roll back functionality). The roller blind or shade 140 may be secured to the first side frame 16 or the second side frame 18, or to or within the electronic box 138, see FIG. 16. The roller blind or shade 140 may be pulled and moved from side to side, using a handle 142, see FIG. 17. Although shown outside the electronic box 138, the roller blind or shade 140 may be placed within so the roller blind or shade reel is hidden within. Alternatively, the roller blind or shade 140 may be secured to the upper frame 12 and lower frame 14, see FIG. 18. The roller blind or shade 140 may be pulled and moved from top to bottom, using a handle 142, see FIG. 19. Both the cover or drape 122 and the roller blind or shade 140 are made of a UV filtering material or material that prevents the transmission of UV light passage.

Referring to FIG. 20, the UV light equipment rack 10 is shown with a plurality of baseball helmets 144 stored therein. The control panel 121 is shown with a power on button 146, power off button 148, and touchscreen display panel 150. The in-use warning system includes a plurality of colored LED lights 152 (green lights for when UV lights are not turned on/red lights for when UV lights are turned on) as part of the electronic box 138 or secured within the second enclosed side wall or surface 136.

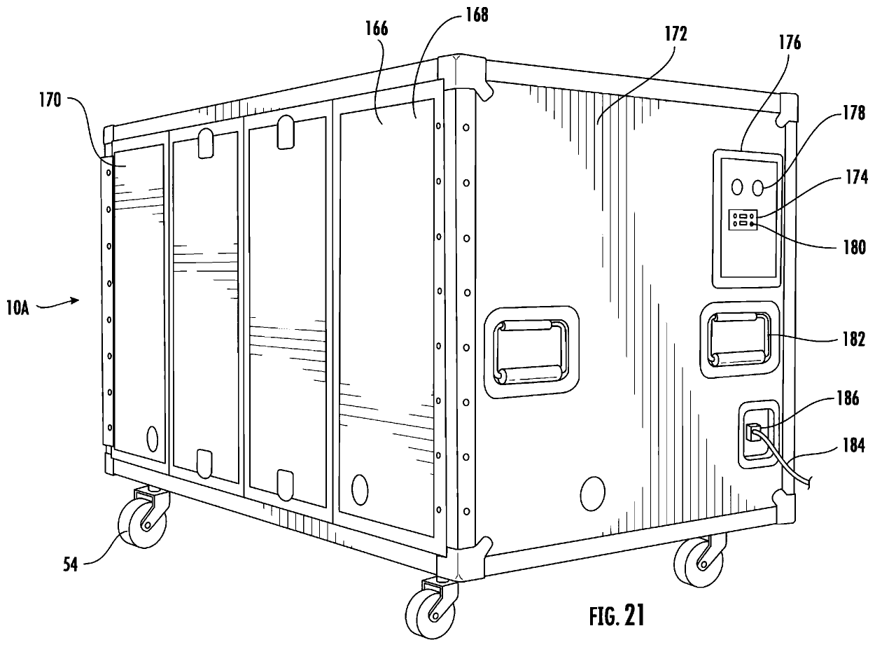

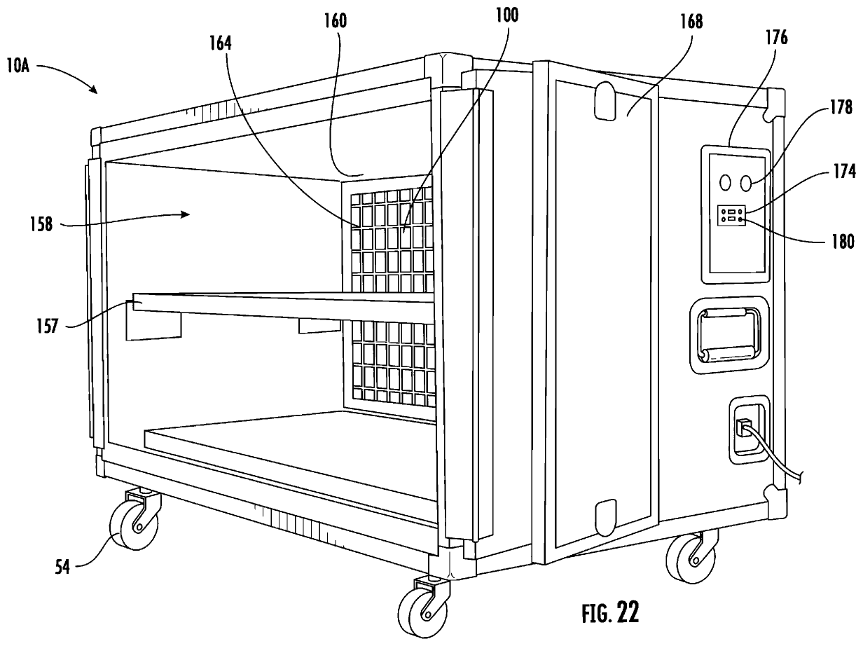

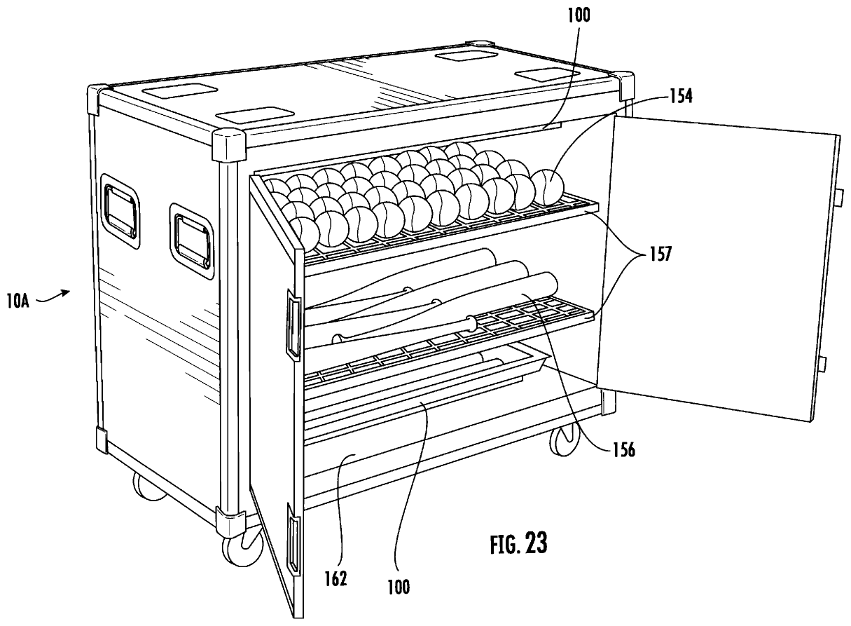

Referring to FIGS. 21-24, smaller embodiments of the UV light equipment rack 10 are shown. Each of these embodiments may include any of the features or structures as described above. Referring specifically to FIGS. 21-23, the UV light equipment rack 10 is configured as a shorter version of any of the UV light equipment racks 10 described above and referred to specifically as UV light equipment rack 10A. The UV light equipment rack 10A is configured to disinfect smaller types of equipment, such as one or more baseballs 154 or baseball bats 156; preferably resting on shelves 157, see FIG. 23 using the UV light panels 100. The UV light panels 100 may be placed within an interior 158, either along a back wall 160 (FIG. 22) or a bottom wall 162 (FIG. 23). If along the back wall 160, a protective grate 164 may be placed in front of the UV light panels 100. The front wall 166 of the UV light equipment rack 10A may contain two hinged panels 168 and 170, acting as doors. A side wall 172 may include a control panel 174 having a power on button 176, a power off button 178, and a touchscreen display panel 180. A pair of handles 182 may be used to move the UV light equipment rack 10A from one location to another location. A power cord 184 and power cord receptacle 186 may be used to supply power to the UV light equipment rack 10A.

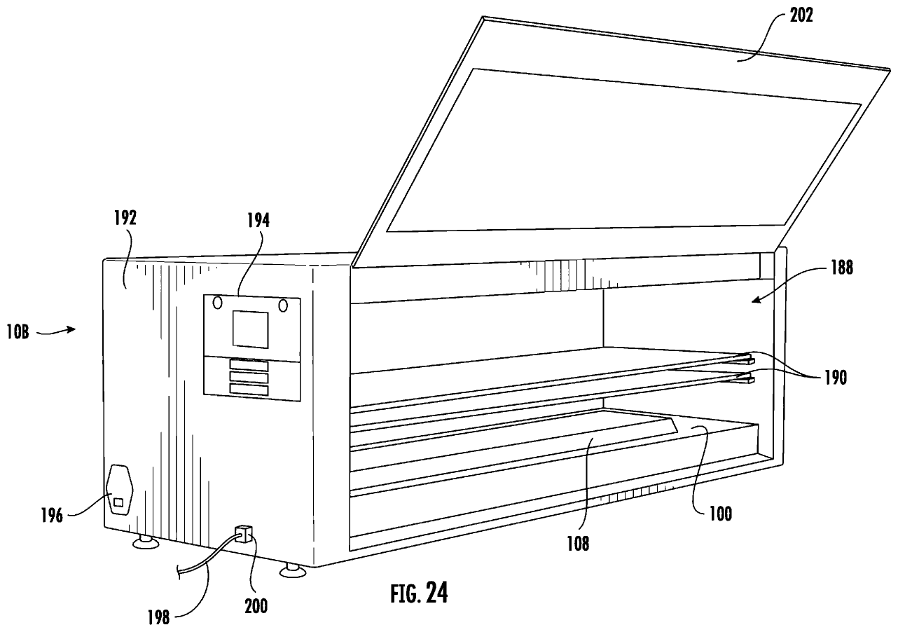

Referring to FIG. 24, a tabletop version of the UV light equipment rack 10, referred to specifically as the UV light equipment rack 10B is illustrated. The UV light equipment rack 10B is designed to disinfect everyday items, such as laptop computers, phones, jewelry, etc., and includes an interior 188 having one or more UV light panels 100 and shelving 190. A side wall 192 may include a control panel 194, a power on/off button 196, and a power cord 198 and power cord receptacle 200 for supplying power to the UV light equipment rack 10B. A door 202 may be used to enclose the interior 188 when in use, i.e. when UV light is turned on.

To aid in the disinfection process, the UV light equipment rack 10 may include one or more ionizer(s) 204, such as the D5 Sterionizer manufactured by Eagle X Pro, see FIG. 1B or FIG. 4A. Preferably, the ionizer 204 is designed to pump ions, such as 10 billion ions per second, into the UV light equipment rack 10 without producing ozone. The positive and negative ions attach to microbes, such as virus/bacteria, breaks them down and rendering them unable to infect. The ionizer 204 maybe be placed and secured to any portion of the UV light equipment rack 10.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A UV light equipment rack configured to disinfect multiple pieces of equipment comprising:
an interior portion defined by an upper structure, a lower structure, a first side structure, and a second side structure;
an equipment holding assembly configured to receive and hold one or more types of equipment, and
a UV light panel configured to emit ultraviolet light arranged to emit UV light upon said equipment holding assembly.

2. The UV light equipment rack configured to disinfect multiple pieces of equipment according to claim 1, comprising at least one holding/disinfecting zone.

3. The UV light equipment rack configured to disinfect multiple pieces of equipment according to claim 1, comprising at least two holding/disinfecting zones.

4. The UV light equipment rack configured to disinfect multiple pieces of equipment according to claim 1, comprising at least three holding/disinfecting zones.

5. The UV light equipment rack configured to disinfect multiple pieces of equipment according to claim 1, wherein said equipment holding assembly comprises one or more equipment holding members configured to hold equipment having a rounded surface.

6. The UV light equipment rack configured to disinfect multiple pieces of equipment according to claim 6, wherein said equipment is a helmet or ball.

7. The UV light equipment rack configured to disinfect multiple pieces of equipment according to claim 1, wherein said UV light panel emits UV-C light.

8. The UV light equipment rack configured to disinfect multiple pieces of equipment according to claim 1, further including a visual warning device operating when said UV light equipment rack is emitting UV-C light.

9. The UV light equipment rack configured to disinfect multiple pieces of equipment according to claim 11, further including an audio safety warning device.

10. The UV light equipment rack configured to disinfect multiple pieces of equipment according to claim 1, wherein said UV light equipment rack is configured to stop operating when a person or animal enters a field of the radiation.

11. The UV light equipment rack configured to disinfect multiple pieces of equipment according to claim 10, further including one or more occupancy sensors configured to immediately shut off said UV light equipment rack if a person or animal is within a disinfection range while said UV light equipment rack is in operating position.

12. The UV light equipment rack configured to disinfect multiple pieces of equipment according to claim 1, further including a front wall.

13. The UV light equipment rack configured to disinfect multiple pieces of equipment according to claim 12, wherein said front wall is configured to slidably move in a vertical direction or a horizontal direction.

14. The UV light equipment rack configured to disinfect multiple pieces of equipment according to claim 1, further including a back wall and a front wall, thereby fully enclosing said interior.

15. The UV light equipment rack configured to disinfect multiple pieces of equipment according to claim 1, further including a cover sized and shaped to enclose said upper structure, said lower structure, said first side structure, and said second side structure.

16. The UV light equipment rack configured to disinfect multiple pieces of equipment according to claim 1, wherein said UV light equipment rack is configured to operate via wireless technology.

17. The UV light equipment rack configured to disinfect multiple pieces of equipment according to claim 16, wherein said wireless technology is WiFi, RF, or Bluetooth.

18. The UV light equipment rack configured to disinfect multiple pieces of equipment according to claim 1, wherein said UV light equipment rack is configured to communicate with a smart phone or a computer tablet.

19. The UV light equipment rack configured to disinfect multiple pieces of equipment according to claim 1, further including an ionizer.
